(19) United States
(12) Patent Application Publication
(10) Pub. No.: US 2025/0380986 A1
(43) Pub. Date: Dec. 18, 2025

(54) METHOD AND APPARATUS FOR PHOTOACOUSTIC-GUIDED ULTRASOUND TREATEMENT FOR PORT WINE STAINS

(71) Applicant: The Regents of the University of California, Oakland, CA (US)

(72) Inventors: Liangzhong XIANG, Oakland, CA (US); Prabodh Kumar PANDEY, Oakland, CA (US)

(73) Assignee: The Regents of the University of California, Oakland, CA (US)

(21) Appl. No.: 19/107,534

(22) PCT Filed: Aug. 29, 2023
(86) PCT No.: PCT/US2023/073118

§ 371 Date: Feb. 27, 2025

Related U.S. Application Data
(60) Provisional application No. 63/403,278, filed on Sep. 1, 2022.

(51) Int. Cl. A61B 18/20; A61B 5/00; A61B 5/02; A61B 18/00

(52) U.S. Cl. CPC A61B 18/20; A61B 5/0095; A61B 5/02007; A61B 2018/00404; A61B 2018/00773; A61B 2018/00994

(57) ABSTRACT

Port wine birthmark, also known as port wine stain (PWS) is a skin discoloration characterized by red/purple patches caused by vascular malformation. In this disclosure, we propose a photoacoustic (PA) guided US focusing methodology for PWS treatment which combines the optical contrast-based selectivity with US penetration to focus the US energy onto the vasculature. The PA signals collected by the transducers when time-reversed and transmitted converge onto the PWS, thus, minimally affecting the neighboring tissue. We performed simulations that mimic realistic transducers and medium properties for this proof of concept study demonstrating the feasibility of the proposed methodology.

### CROSS REFERENCE TO RELATED APPLICATIONS

The present application claims priority to U.S. Provisional Application No. 63/403,278 filed Sep. 1, 2022, the contents of which are incorporated herein by reference in their entirety.

### TECHNICAL FIELD

The present embodiments relate generally to Port wine stains (PWS), photoacoustic (PA)-guided ultrasound (US) and time-reversal (TR).

### BACKGROUND

Port wine birthmark, also known as port wine stain (PWS) is a skin discoloration characterized by red/purple patches caused by vascular malformation. PWS is typically treated by using lasers to selectively destroy the abnormal blood vessels. However, light attenuates quickly in the tissue due to high optical scattering and hence residual abnormal blood vessels deep in the tissue survive and often lead to the resurgence of PWS.

It is against this technological backdrop that the present Applicant sought a technological solution to these and other problems rooted in this technology.

### SUMMARY

The present embodiments relate to a photoacoustic (PA) guided US focusing methodology for PWS treatment which combines optical contrast-based selectivity with US penetration to focus US energy onto the vasculature. In embodiments, the tissue is first irradiated by a nano-second laser pulse and the PA signal thus generated is collected by ultrasonic transducers. These PA signals, when time-reversed, amplified and transmitted, converge onto and destroy the PWS, while affecting the neighboring tissue minimally.

### BRIEF DESCRIPTION OF THE DRAWINGS

These and other aspects and features of the present embodiments will become apparent to those ordinarily skilled in the art upon review of the following description of specific embodiments in conjunction with the accompanying figures, wherein:

FIGS. 1A-1C are example schematic diagrams of (a) laser beam treatment, (b) ultrasound treatment, and (c) PA guided ultrasound therapy according to embodiments.

FIGS. 2A and 2B illustrate example aspects of PA-guided US focusing for PWS treatment according to embodiments.

FIGS. 3A to 3C illustrate example aspects of full and limited-view geometries according to embodiments.

FIG. 4 is a functional block diagram illustrating an example treatment procedure according to embodiments.

FIGS. 5A to 5D illustrate example effects of acoustic attenuation on PA signals according to embodiments.

FIGS. 6A to 6F illustrate example aspects of converging an ultrasound signal to the shape of the blood vessel vasculature according to embodiments.

FIGS. 7A to 7F illustrate example aspects of the effects of element size on a focused US map according to embodiments.

FIGS. 8A to 8F illustrate example aspects of the effects of full and partial-views on the converged US field according to embodiments.

FIGS. 9A to 9F illustrate example aspects of filtering of PA signals according to embodiments.

FIGS. 10A to 10F illustrate example aspects of filtering of PA signals according to embodiments.

FIGS. 11A to 11F illustrate example aspects of ultrasound energy distribution obtained from a ring array of US transceivers according to embodiments.

### DETAILED DESCRIPTION

The present embodiments will now be described in detail with reference to the drawings, which are provided as illustrative examples of the embodiments so as to enable those skilled in the art to practice the embodiments and alternatives apparent to those skilled in the art. Notably, the figures and examples below are not meant to limit the scope of the present embodiments to a single embodiment, but other embodiments are possible by way of interchange of some or all of the described or illustrated elements. Moreover, where certain elements of the present embodiments can be partially or fully implemented using known components, only those portions of such known components that are necessary for an understanding of the present embodiments will be described, and detailed descriptions of other portions of such known components will be omitted so as not to obscure the present embodiments. Embodiments described as being implemented in software should not be limited thereto, but can include embodiments implemented in hardware, or combinations of software and hardware, and vice-versa, as will be apparent to those skilled in the art, unless otherwise specified herein. In the present specification, an embodiment showing a singular component should not be considered limiting; rather, the present disclosure is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present embodiments encompass present and future known equivalents to the known components referred to herein by way of illustration.

### Introduction

Port wine stain (PWS), a skin discoloration characterized by deep red or purple patches, represents the second most common congenital vascular malformation affecting 3-5 of every 1000 infants born (K. M. Updyke and A. Khachemoune, “Port-Wine Stains: A Focused Review on Their Management,” Journal of drugs in dermatology, vol. 16, no. 11, pp. 1145-1151, 1 Nov. 2017). Port wine stains often darken over time and potentially become nodular or hypertrophic when left untreated. Previous therapeutic methods for treating port wine stains include surgical excision, freezing, tattooing, and radiation therapy (A. E. Ortiz and J. S. Nelson, “Port-wine stain laser treatments and novel approaches,” Facial plastic surgery, vol. 28, no. 6, pp. 611-620, 28 Dec. 2012). These methods did not produce satisfactory results and often caused cosmetic scarring. Thus, these methods are outdated and no longer practiced.

Introduced in the 1970s, Argon laser treatment was one of the first effective approaches to PWS therapy (P. McLean, “Family practice: a strategy for survival,” Canadian Family Physician, vol. 26, no. 6-7, 1980). The argon laser emits blue-green light between 488 and 514 nm, which is absorbed more readily by hemoglobin in PWS blood vessels (K. M. Kelly, B. Choi, S. McFarlane, A. Motosue, B. Jung, M. H. Khan, J. C. Ramirez-San-Juan and J. S. Nelson, “Description and analysis of treatments for port-wine stain birthmarks,” Archives of facial plastic surgery, vol. 7, no. 5, pp. 287-294, 2005). As a result of absorbing radiant energy, the vessels heat up, causing thrombosis and destruction. However, it also has the potential to cause irreversible damage to the epidermis as a result of unwanted absorption by melanin. In 1983, the concept of selective photothermolysis was presented (R. R. Anderson and J. A. Parish, “Selective photothermolysis: precise microsurgery by selective absorption of pulsed radiation,” Science, vol. 220, no. 4596, pp. 524-7, 1983) in which the laser wavelength is carefully chosen based on the target chromophore to achieve photon absorption and target heating, while neighboring tissues are mostly unaffected, thus reducing negative consequences such as scarring and dyspigmentation.

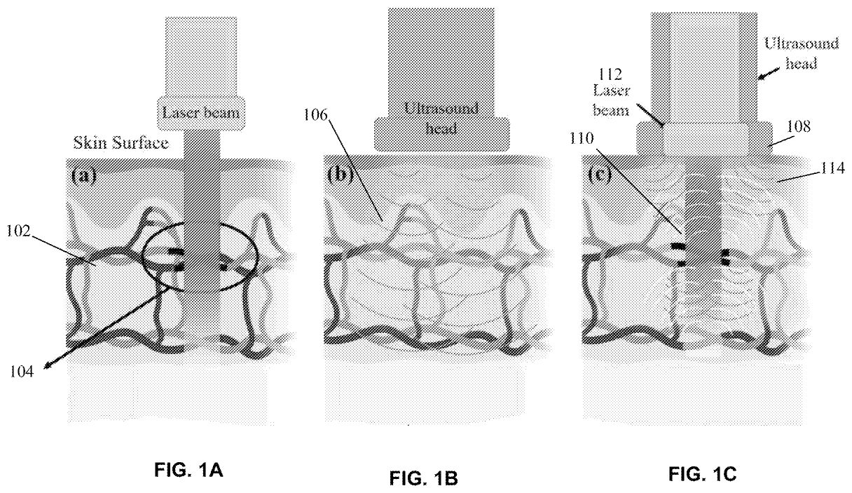

Since the 1980s, PWS birthmarks are commonly treated using a pulsed-dye laser (PDL) in conjunction with epidermal cooling (S. A. Sharif, E. Taydas, A. Mazhar, R. Rahimian, K. M. Kelly, B. Choi and A. J. Durkin, “Noninvasive clinical assessment of port-wine stain birthmarks using current and future optical imaging technology: a review,” The British journal of dermatology, vol. 167, no. 6, pp. 1215-23, 2012; L. A. Brightman, R. G. Geronemus and K. K. Reddy, “Laser treatment of port-wine stains,” Clinical, cosmetic and investigational dermatology, vol. 8, pp. 27-33, 2015). Pulsed dye lasers operating at 577 nm-600 nm wavelengths are preferentially absorbed by hemoglobin in the PWS blood vessels causing thermal damage and thrombosis (J. S. Nelson, T. E. Milner, B. Anvari, B. S. Tanenbaum, S. Kimel, L. Svaasand and S. L. Jacques, “Dynamic epidermal cooling during pulsed laser treatment of port-wine stain. A new methodology with preliminary clinical evaluation,” Archives of dermatology, vol. 131, no. 6, pp. 695-700, 1995). Nonetheless, PDLs have their shortcomings. As shown in FIG. 1A, the penetration depth of PDLs in tissue is limited as indicated by 104 due to high optical scattering and hence deeper capillaries 102 remain untreated after the PDL therapy (S. Khandpur and V. K. Sharma, “Assessment of Efficacy of the 595-nm Pulsed Dye Laser in the Treatment of Facial Port-Wine Stains in Indian Patients,” Dermatologic Surgery, vol. 42, no. 6, pp. 717-726, 2016). These untreated capillaries 102 lead to the growth of new capillaries and hence the resurgence of PWS. Furthermore, increasing the intensity to generate larger vascular damage has the potential to cause dyspigmentation, especially in dark-skinned patients who have more epidermal melanin and reactive fibroblast responses (L. Torezan, “Lasers in Pigmented Skin,” Pigmented Ethnic Skin and Imported Dermatoses, vol. 1, no. 47, pp. 519-527, 2018). The more melanin present, the more likely it is that melanin will absorb the energy and compete with other chromophores, lowering the treatment's efficacy. Still further, efficiently cooling the skin surface (cryogen spray, cold air, cooled gel, ice packs) is necessary to reduce pain during treatment and avoid adverse effects.

Photodynamic therapy (PDT) as a vascular-targeted method for PWS treatment has shown to be potentially effective (Y. Zhao, P. Tu and G. Zhou, “Hemoporfin Photodynamic Therapy for Port-Wine Stain: A Randomized Controlled Trial,” PloS, vol. 11, no. 5, p. 156219, 2016; A. T. Khalaf, Y. Sun, F. Wang and M. Sheng, “Photodynamic Therapy Using HMME for Port-Wine Stains: Clinical Effectiveness and Sonographic Appearance,” BioMed Research International, vol. 2020, pp. 1-7, 2020; K.-H. Yuan, Q. Li, W.-L. Yu and Z. Huang, “Photodynamic therapy in treatment of port wine stain birthmarks—recent progress,” Photodiagnosis and photodynamic therapy, vol. 6, no. 3, pp. 189-94, 2009). PDT is a treatment that combines a photosensitizer (typically injected intravenously), light, and oxygen to trigger a photochemical reaction that produces highly reactive singlet oxygen molecules that can cause cell death through apoptosis, necrosis, or autophagy. The treatment can damage vascular endothelial cells selectively, coagulate blood vessels, cause fibrosis, and facilitate the removal of the skin's distorted enlarged capillary network. However, there are risks of infection, thick scabs, and scar formation due to excessive PDT sessions or improper post-operative care (L.-C. Zhang, J. Yang, Y.-B. Huang and Z. Huang, “Post treatment care in photodynamic therapy (PDT) of large facial port-wine stain (PWS) birthmarks,” Photodiagnosis and photodynamic therapy, vol. 36, no. 102604, 2021). Moreover, phototoxicity (due to the photosensitizer drug) can cause short and long-term side effects such as blisters and pigmentary change (K.-H. Yuan, J.-H. Gao and Z. Huang, “Adverse effects associated with photodynamic therapy (PDT) of port-wine stain (PWS) birthmarks,” Photodiagnosis and photodynamic therapy, vol. 9, no. 4, 2012).

According to a 2019 peer-reviewed article with 65 studies comprising 6207 PWS patients, only 21% of patients experienced 75-100% of skin clearance (M. I. van Raath, S. Chohan, A. Wolkerstorfer, C. van der Horst, G. Storm and M. Heger, “Port wine stain treatment outcomes have not improved over the past three decades,” J Eur Acad Dermatol Venereol, vol. 33, no. 7, p. 1369-1377, 2019). In spite of significant technological advancements and pharmacological therapies, the efficacy of PWS therapy has not increased in recent years. Across the 65 studies spanning the past three decades, there was no evidence of any upward trend in mean clearance over time. Although new technologies enter and mature the clinical setting, the proportion of patients with 75-100% clearance has not increased. In summary, only a few studies in the last 30 years have been able to match or exceed the findings reported in the 1980s and early 1990s with the 577 and 585 nm PDLs.

Among other things, the present Applicant has recognized that ultrasound waves have been demonstrated to be effective in treating a variety of wounds and PWS. As shown by FIG. 1B, therapeutic ultrasound (US) is a physical way of delivering non-ionizing radiation into the tissues in the form of mechanical sound waves 106, which cause tissue heating (S. A. Alkahtani, P. S. Kunwar, M. Jalilifar, S. Rashidi and A. Yadollahpour, “Ultrasound-based Techniques as Alternative Treatments for Chronic Wounds: A Comprehensive Review of Clinical Applications,” Cureus, vol. 9, no. 12, p. 1952, 2017; A. Yadollahpour, M. Jalilifar, S. Rashidi and Z. Rezaee, “Ultrasound Therapy for Wound Healing: A Review of Current Techniques and Mechanisms of Action,” Journal of Pure and Applied Microbiology, vol. 8, no. 5, pp. 4071-4085, 2014). The key advantages of ultrasound wound treatment include deep penetration into the wound and minimal risk of negative effects. Ultrasound is a frequently-used modality to image PWS lesions (J. Buch, P. Karagaiah, P. Raviprakash, A. Patil, G. Kroumpouzos, M. Kassir and M. Goldust, “Noninvasive diagnostic techniques of port wine stain,” Journal of cosmetic dermatology, vol. 20, no. 7, pp. 2006-2014, 2021; A. Troilius, G. Svendsen and B. Ljunggren, “Ultrasound investigation of port wine stains,” Acta dermato-venereologica, vol. 80, no. 3, pp. 196-9, 2000), yet very little literatures can be found on using ultrasound as a therapeutic treatment for PWS. In 2020, Kwiek et al. utilized high-intensity focused ultrasound (HIFU) treatment to improve soft tissue hypertrophy which can be associated with PWS (B. Kwiek, Ł. Paluch, C. Kowalewski and M. Ambroziak, “Facial Hypertrophic Port-Wine Stain Treatment Combining Large Spot 532 nm Laser, High-Intensity Focused Ultrasound and Traction Threads,” Dermatologic Surgery, vol. 46, no. 7, pp. 988-990, 2020). The most notable shortcoming regarding ultrasound therapy for PWS treatment is lack of selectivity. Ultrasound therapy does not differentiate PWS-affected vasculature 102 from surrounding healthy tissue, resulting in the entire area being irradiated. To prevent this issue, a new technique is needed to target aberrant vasculature.

The present Applicant has further recognized that several past studies utilized PA guidance for US focusing (M. K. A. Singh and W. Steenbergen, “Photoacoustic-guided focused ultrasound (PAFUSion) for identifying reflection artifacts in photoacoustic imaging,” Photoacoustics, vol. 3, no. 4, pp. 123-131, 2015; M. K. A. Singh, M. Jaeger and M. Frenz, “In vivo demonstration of reflection artifact reduction in photoacoustic imaging using synthetic aperture photoacoustic-guided focused ultrasound (PAFUSion),” Biomedical optics express, vol. 7, no. 8, pp. 2955-72, 2016; A. Prost, A. Funke and M. Tanter, “Photoacoustic-guided ultrasound therapy with a dual-mode ultrasound array,” Journal of biomedical optics, vol. 17, no. 6, 2012). However, these studies used the PA guidance to focus the US onto a focal point. The time-reversal-based focusing as demonstrated in this manuscript facilitates US focusing onto the optical absorption map. The proposed strategy could also be used with existing methodology of ultrasound sonication using microbubbles for rupturing blood vessels (K. Doucette, J. O'Malley and P. White, “A Novel Application of Focused Ultrasound for the Treatment of Port Wine Stain Birthmarks,” National Library of Medicine, vol. 2019, no. 1, 2019; N. Hosseinkhah, H. Chen and T. J. Matula, “Mechanisms of microbubble-vessel interactions and induced stresses: A numerical study,” The Journal of the Acoustical Society of America, vol. 134,no. 3, pp. 1875-85, 2013; R. Singh, J. Jo and M. Riegel, “The feasibility of ultrasound-assisted endovascular laser thrombolysis in an acute rabbit thrombosis model,” Medical physics, vol. 48, no. 8, pp. 4128-4138, 2021), as well as for improved permeability for intravascular delivery of gene therapy and medications (C. Y. Lin, H. C. Tseng and H. R. Shiu, “Ultrasound sonication with microbubbles disrupts blood vessels and enhances tumor treatments of anticancer nanodrug,” International journal of nanomedicine, vol. 7, pp. 2143-52, 2012). This methodology can also assist other targeted ultrasound therapeutic techniques aimed at wound healing and cancer therapy (P. Lai, C. Tarapacki and W. Tran, “Breast tumor response to ultrasound mediated excitation of microbubbles and radiation therapy in vivo,” Oncoscience, vol. 3, no. 3-4, pp. 98-108, 2016; Y. J. Ho, S. W. Chu and E. C. Liao, “Normalization of Tumor Vasculature by Oxygen Microbubbles with Ultrasound,” Theranostics, vol. 9, no. 24, pp. 7370-7383, 2019; A. K. W. Wood and C. M. Sehgal, “A review of low-intensity ultrasound for cancer therapy,” Ultrasound in Medicine & Biology, vol. 41, no. 4, pp. 905-28, 2015; G. ter Haar, “Therapeutic applications of ultrasound,” Progress in biophysics and molecular biology, vol. 93, no. 1-3, pp. 111-29, 2007).

According to certain aspects, the present embodiments relate to a novel treatment technique for PWS treatment which utilizes PA guided US to focus onto the blood vessels. As shown in FIG. 1C, the PWS 102, when excited by a pulsed laser 112, generates PA waves 110, which PA waves can be sensed using ultrasonic transducers 108 placed around the diseased tissue. These PA signals 110 carry the information of the location and shape of the vasculature. These PA signals when time-reversed by processing circuitry and transmitted from the transducers 108, converge the US energy 114 onto the PWS itself, sparing the healthy neighboring tissues.

### Theory

The generation of photoacoustic waves following optical energy deposition (OED) in the tissue under the assumptions of thermal confinement and zero acoustic attenuation is based on the photoacoustic equation (L. V. Wang and H.-i. Wu, Biomedical Optics: Principles and Imaging, Hoboken: John Wiley & Sons, 2012):

$${\partial }^{2} p ( {r}^{\to } , t ) / \partial {t}^{2} - {v}^{2} {∇}^{2} p ( {r}^{\to } , t ) = Γ H ( {r}^{\to } ) \partial δ ( t ) / \partial t , \qquad ( 1 )$$

where Γ(=ν²β/Cₚ) is the Gruneisen parameter, ν is the speed of sound, β is the volumetric expansion coefficient, Cₚ is the specific heat at constant pressure and H is the OED distribution. The pressure fields p (r^{→},t) can be sensed using ultrasonic transducers placed on a detection grid around the region of interest till time T, long enough to ensure that the pressure fields vanish at each of the detectors for t>T.

The impulse response of acoustic detection h^{AD} is the pressure signal sensed by a detector due to a Dirac delta source located at r^{→}₀ can be expressed as

$${h}^{AD} ( {r}_{0}^{\to } , t ) = \int G ( {r}^{\to } , t ; {r}_{0}^{\to } , t ) W ( {r}^{\to } ) d {r}^{\to } \qquad ( 2 )$$

with G(r^{→},t;r^{→}₀,t) being the Green's function corresponding to Eq. 1, representing the pressure field at r^{→} due to the Dirac source located at r^{→}₀ and W(r^{→}) being the detector apodization (P. Warbal, M. Pramanik and R. Saha, “Impact of sensor apodization on the tangential resolution in photoacoustic tomography,” Journal of the Optical Society of America. A, Optics, image science, and vision, vol. 36, no. 2, pp. 245-252, 2019). The integral in Eq. 2 represents the weighted integral over the transducer surface. Similarly, the impulse response of acoustic transmission h_{AT} is the pressure field at r^{→}₀ due to Dirac delta transmission sources on the transducer surface and can be expressed as:

$${h}^{AT} ( r {0}^{\to } , T ) = \int G ( {r}^{\to } 0 , t ; {r}^{\to } , t ) W ( {r}^{\to } ) d {r}^{\to } \qquad ( 3 )$$

with G(r^{→}₀,t;r^{→},t) being the Green's function corresponding to Eq. 1, representing the pressure field at r^{→}₀ due to the Dirac source located at r^{→}. Using the reciprocity theorem, it can be concluded that the detection and transmission impulse responses are the same, i.e. h^{AT}(r0^{→},t)=h^{AD}(r0^{→},t)=h^{A}(r0^{→},t).

The signals collected by the transducers can be processed (e.g. time-reversed) to produce focusing information for subsequent US treatment by the transducers using any processing circuitry adapted as follows.

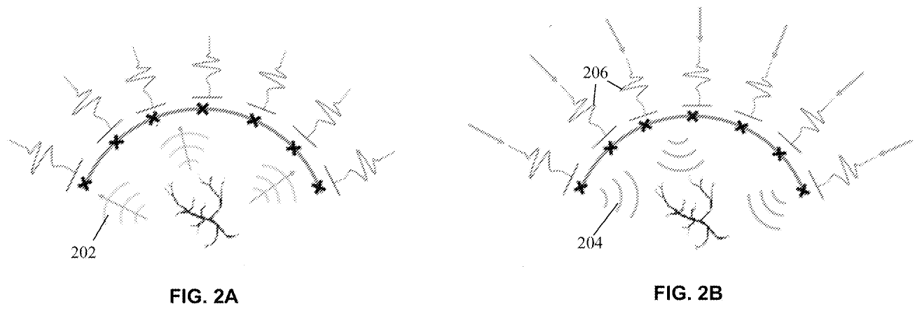

The pressure signal collected at ith detector (e.g. shown by signals 202 in FIG. 2A), located at r^{→}ᵢ due to a pressure source at r^{→}₀ can be expressed as the convolution (denoted by * sign) between the electrical and acoustic responses (h^{E}, h^{A} respectively) of the detector.

$${p}_{i} ( t ) = {h}_{i}^{E} ( t ) * {h}_{i}^{A} ( {r}^{\to } 0 , t ) \qquad ( 4 )$$

The pressure signal when time-reversed is given as

$${p}_{i} ( T - t ) = {h}_{i}^{E} ( T - t ) * {h}_{i}^{A} ( {r}^{\to } 0 , T - t ) \qquad ( 5 )$$

The pressure field at r^{→}₀ generated due to the ith transducer can be given as the convolution of the combined electrical and acoustic responses with the time-reversed pressure signal, i.e.

$$p ( {r}^{\to } 0 , t ) = {h}_{i}^{E} ( T - t ) * {h}_{i}^{A} ( {r}^{\to } 0 , T - t ) * {h}_{i}^{E} ( t ) * {h}_{i}^{A} ( {r}^{\to } 0 , t ) \qquad ( 6 )$$

The total pressure field due to the contribution from the time-reversed fields transmitted by all the (say total N) transducers is thus expressed as

$$p ( {r}^{\to } 0 , t ) = \sum {h}_{i}^{E} ( T - t ) * {h}_{i}^{A} ( {r}^{\to } 0 , T - t ) * {h}_{i}^{E} ( t ) * {h}_{i}^{A} ( {r}^{\to } 0 , t ) \qquad ( 7 )$$

The time-reversed acoustic fields at r^{→}₀ due to each of the transducers attain their maximum at t=T, thus constructively interfering to produce focused US field at r^{→}₀. Since this is true for an arbitrarily chosen location r^{→}₀, the idea as shown in FIG. 2B, can be extended to focus the transmitted US signals 204 from the transducers 206 onto the initial pressure source map H (r^{→}) at time t=T (M. Fink, “Time reversal of ultrasonic fields. I. Basic principles,” IEEE transactions on ultrasonics, ferroelectrics, and frequency control, vol. 39, no. 5, pp. 555-566, 1992).

While in principle, the concept of time-reversal holds for acoustically non-attenuating media, it does reasonably well for weakly absorbing soft-tissue-like media. Additional sources for inaccuracies in time-reversed fields are finite detection bandwidth and size, detector spacing and limited-view availability for ultrasound collection whose effect will be demonstrated in this paper.

### Numerical Studies

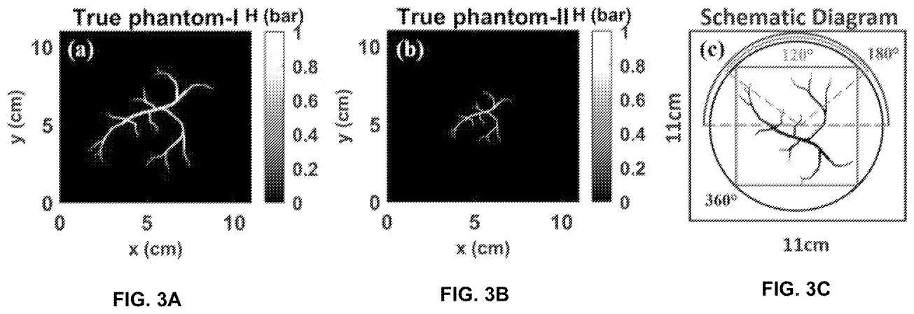

The present Applicant performed numerical studies using the k-wave toolbox (B. E. Treeby and B. T. Cox, “k-Wave: MATLAB toolbox for the simulation and reconstruction of photoacoustic wave fields,” Journal of Biomedical Optics, vol. 15, no. 2, 2010) to demonstrate the feasibility of time-reversal guided ultrasound treatment of PWS. Two phantoms mimicking a blood vessel network (of different sizes as shown in FIGS. 3A and 3B) were chosen as the pressure sources following absorption of the optical energy from a pulsed laser with ˜550-600 nm wavelength which is predominantly absorbed by hemoglobin in PWS blood vessels. Assuming a uniform illumination of the considered structure, a fluence of ˜1.5 mJ/cm' and blood absorption coefficient of ˜350/cm yields 500 mJ/cm³ of absorbed energy density (M. Keijzer, J. W. Pickering and M. J. Gemert, “Laser beam diameter for port wine stain treatment,” Lasers in Surgery and Medicine, vol. 11, no. 6, pp. 601-605, 1991). This leads to ˜1 bar rise in the local pressure inside the blood vessel. For setting up the simulation in the k-wave toolbox, the region of interest is considered as a square with side length of 11 cm as shown in FIG. 3C.

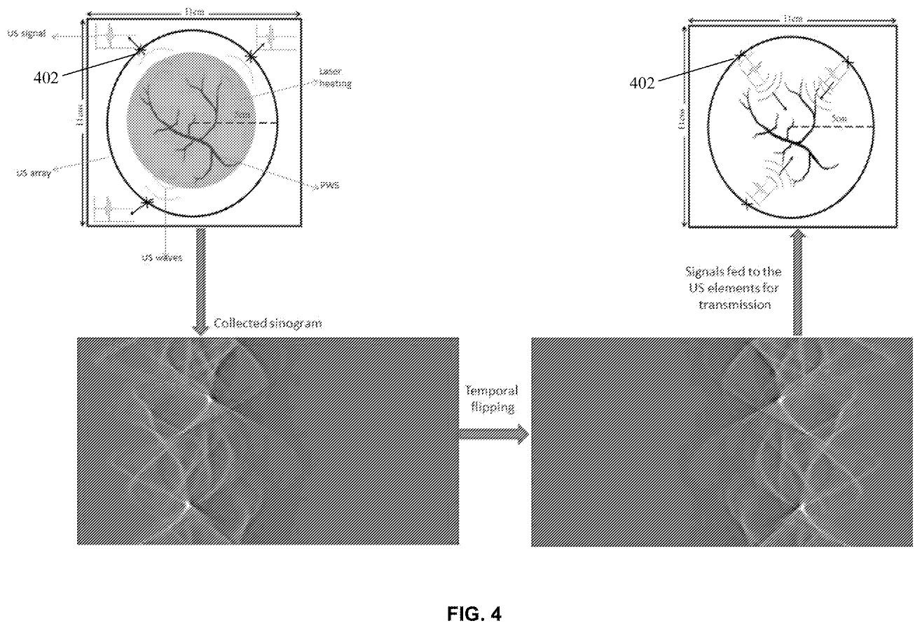

As shown in FIG. 4, for the studies carried out with phantom 1, the computation region was discretized into 472×472 regular grid points with h˜0.23 mm grid resolution. This grid resolution supports the maximum frequency of ν2h/˜3.22 MHz for the field propagation. The pressure signals were collected at 20 MHz sampling frequency (satisfying the Nyquist criterion (A. V. Oppenheim, R. W. Schafer and J. R. Buck, Discrete Time Signal Processing, New Jersey: Prentice hall, 2001): fₛ≥2fₘₐₓ) for 75 microseconds. Owing to the thinner structures, for simulations with phantom 2, the grid resolution was chosen to be ˜0.12 mm and the sampling frequency was chosen to be 40 MHz. In all the simulations, the blood vessel phantom was placed at the center of the computational domain. The speed of sound throughout the medium was chosen as ν=1500 m/s. The blood vessel region was surrounded by a number of detectors 402 placed on a circle with 5 cm radius. The number of transducers as well as the collection coverage angles and transducer bandwidths were varied to study their effect on the converging time-reversed fields.

FIG. 4 illustrates an example numerical simulation (PA excitation, resulting in a collected sinogram, time-reversal, and signals fed back to transducers 402 for guided US focusing) according to embodiments. We also incorporated the frequency dependent acoustic attenuation of the fields while propagating in the soft tissue which follows the frequency power law given as (H. Ammari, E. Bretin and V. Jugnon, “Photoacoustic Imaging for Attenuating Acoustic Media,” Mathematical Modeling in Biomedical Imaging II, pp. 57-84, 2012; B. E. Treeby, “Acoustic attenuation compensation in photoacoustic tomography using time-variant filtering,” Journal of biomedical optics, vol. 18, no. 3, 2013):

$$α ( ω ) = {α0ω}^{y}$$

where α(ω) denotes the acoustic attenuation (unit: dB/m) at the angular frequency ω (unit: rad/s), α₀ is the power law prefactor (unit: dB (rad/s)_{-y} m⁻¹) with y being the power law exponent (typically 1≤y≤2 in soft tissue). Embodiments use y=1.5 and α₀=0.5 dB (rad/s)^{-1.5} m⁻¹), consistent with the acousto-mechanical properties of human tissue.

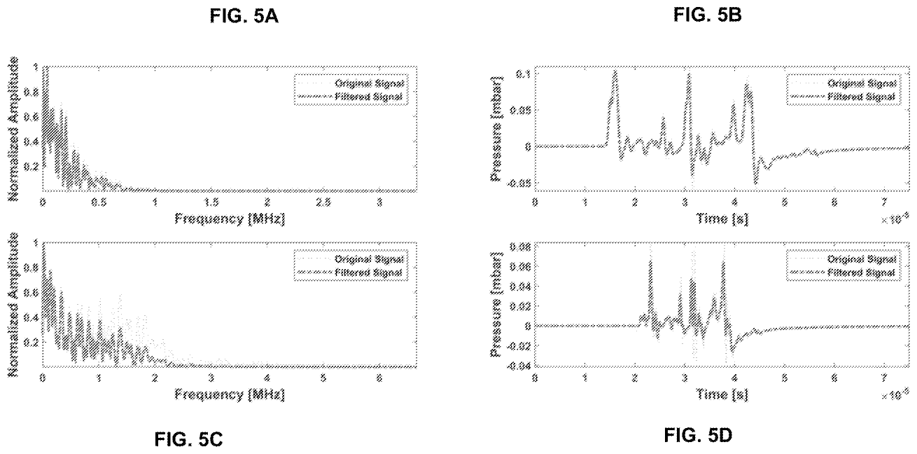

The effect of acoustic attenuation on the broadband PA signals generated by phantom 1 and 2 are demonstrated in FIGS. 5A to 5D. More particularly, these figures illustrate the effects of acoustic attenuation on the PA signals: the attenuated (dashed) and un-attenuated (dotted) frequency-domain signals (FIGS. 5A and 5C) and time-domain signals (FIGS. 5B and 5D) generated from phantom 1 and 2, respectively. As evident from Eq. 2, a positive value of the power law exponent y means the higher frequency components of the PA signals will be attenuated more as compared to the lower frequency ones. The thickness of the structures in the blood vessel in phantom 1 is larger than those in phantom 2. Therefore, while the frequency content of phantom 1 is limited to ˜0.75 MHz, it extends to ˜3 MHz for phantom 2. Since higher acoustic frequencies are more attenuated in the tissue, the difference between the amplitudes of the original and attenuated time-domain PA signals is more significant for phantom 2 as compared to phantom 1.

To model the finite transducer shape and directivity, each of the transducer elements was divided into multiple sub-detection points and the detected signal at the element is considered as the weighted average of the signals collected at sub-detection points. Employed was the Gaussian apodization for computing the weighted average (Id.):

$$p ( r {s}^{\to } , t ) = \int p ( {r}^{\to } , t ) W ( {r}^{\to } ) d {r}^{\to } \qquad ( 9 )$$

where p (rS^{→→→→},t) denotes the pressure field recorded by a detector centered at rS^{→→→→}, p (r^{→},t) is the pressure field at a location r^{→} (on the surface of detector S) and W(r^{→}) represents the Gaussian weights. The transducer apodization is considered while sensing as well as transmitting the US signals. Once the transducers are fully defined, the detection simulation is run by calling the function ‘kspaceFirstOrder2D’, the modeled signals are then contaminated with white Gaussian noise to achieve 2 dB SNR. The simulated photoacoustic signals are broadband and have frequency components from 0 to fs/2; fs being the sampling frequency. However, the detection system is typically bandlimited and for an accurate experimental realization, the simulated PA signals are convolved with the transducer's frequency response mimicked as a Gaussian characterized by the central frequency and −6 dB bandwidth.

In simulations, this has been performed using the ‘gaussianFilter’ from the toolbox. After the (simulated) PA signals were collected at each of the transducers, the transducers were then switched to the transmission mode, i.e. each transducer acts as a temporally varying pressure source. Each transducer was modeled to transmit the time-reversed (temporally flipped) PA signal it collected and the temporal evolution of these pressure fields was observed to converge to the PWS model blood vessel. The linearity of the acoustic propagation equation implies that higher pressure strengths in the PWS can be achieved by simply amplifying the US signals transmitted by the transducers. PWSs typically affect the head and neck (C. G. Lian, L. M. Sholl and L. R. Zakka, “Novel genetic mutations in a sporadic port-wine stain,” JAMA dermatology, vol. 15, no. 12, pp. 1336-40, 2014), where collection of PA signals all around the region of interest is not feasible. Therefore, simulations have also been performed to study the efficacy of the treatment method with limited collection—transmission views—180° and 120°, as shown in FIG. 3C.

### Example Results and Discussion

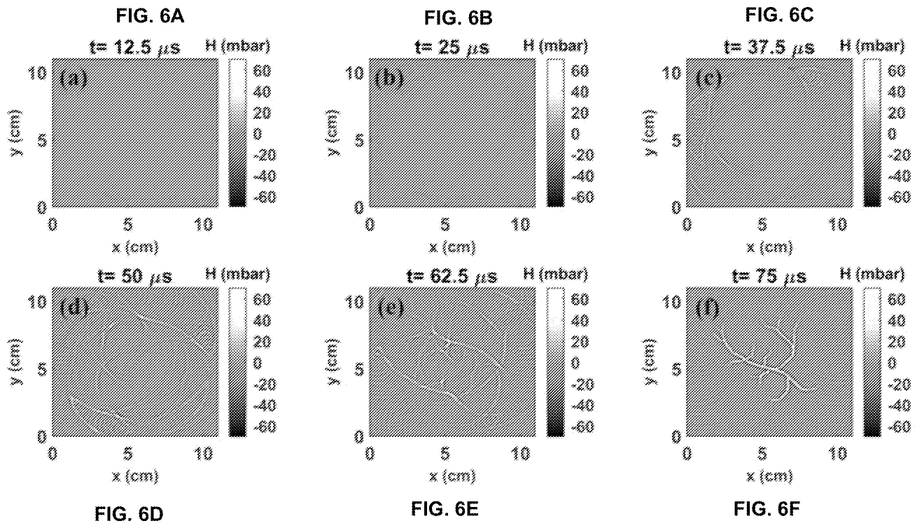

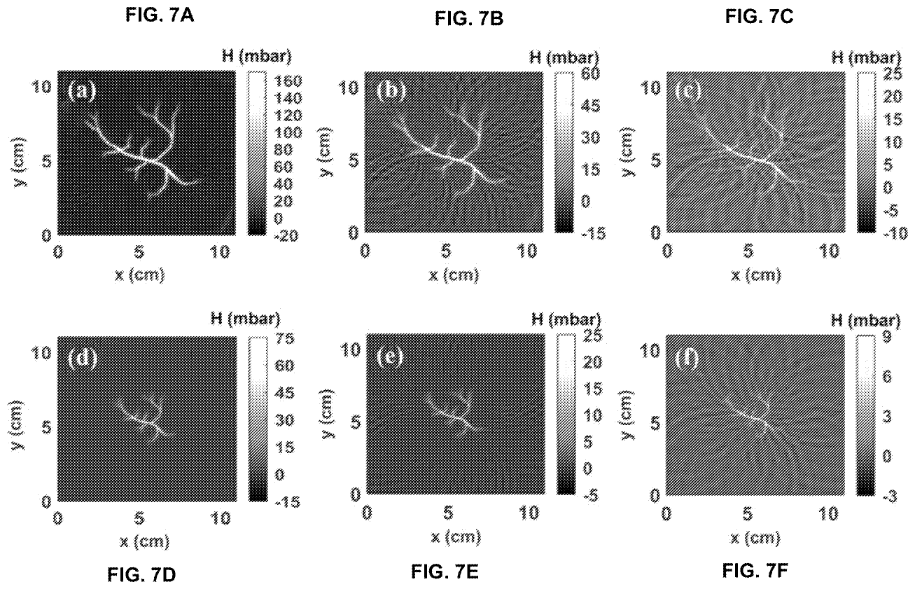

The PA signals were collected by the transducers for T=75 μs. As discussed above, the PA signals when time-reversed and transmitted from each elements of the transducer grid, converge on to the blood vessel. FIGS. 6A to 6F illustrate temporal evolution of the US signals emitted by the transducers for phantom 1 for 128 elements (fc=0.5 MHz, 80% bandwidth), full-view circular array. Spacing between two consecutive transducers also governs the performance of the proposed method. As demonstrated in FIGS. 6A to 6F, the US waves emitted from the transducers converge onto the whole blood vessel when a 128 element grid is employed. This is also shown in FIGS. 7A and 7D for phantoms 1 and 2, respectively. However, when the number of elements is reduced to 64, the central parts of the blood vessel receive higher US energy as compared to its branches far from the center as shown in FIGS. 7B and 7E. When the number of elements on the grid is further reduced to 32, the US waves converge on to an approximately ˜2 cm region of the blood vessel as shown in FIGS. 7C and 7F. The off-center branches receive lower US and US energy radiates to the neighboring tissue. Therefore, in order to treat the PWS, higher number of elements is desired on the transducer grid. This will improve the focusing of the transmitted US waves on to the target blood vessels.

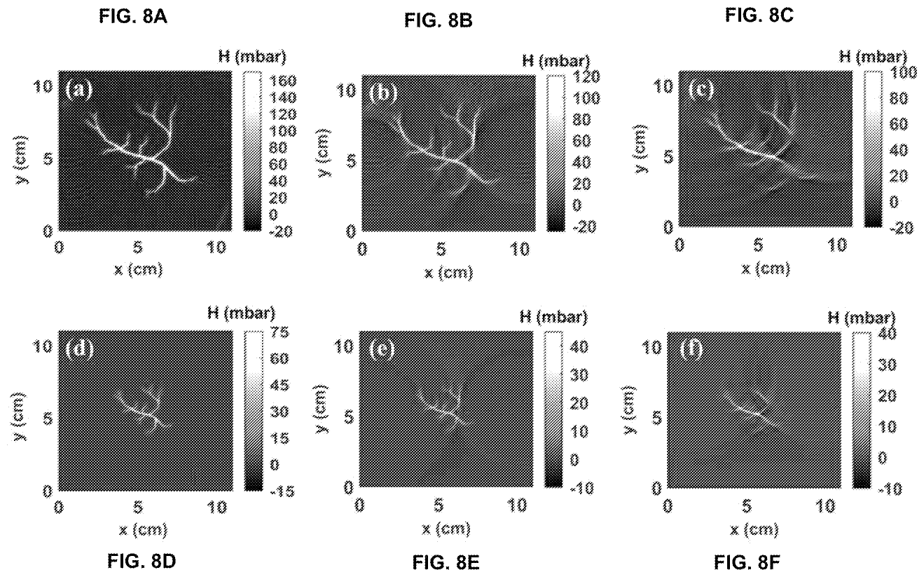

Placing transducers all around the diseased tissue may not be possible as PWSs are commonly located on the head and neck. Moreover, in several scenarios, the path of US propagation is hindered by bones which the US cannot penetrate through (J. Smith and J. T. Finnoff, “Diagnostic and interventional musculoskeletal ultrasound: part 1. Fundamentals,” PM & R: the journal of injury, function, and rehabilitation, vol. 1, no. 1, pp. 64-75, 2009). In such cases, the US grid has limited-view/coverage. Simulations have been performed to study the effect of the limited-view on the focusing of the transmitted US waves. FIGS. 8A to 8F demonstrate example US focusing for full-view (128 elements, 360°), half-view (65 elements, 180°) and one-third-view (44 elements, 120°) settings with fixed spacing between elements. As expected, the transmitted US converges best on the phantom 1 and 2 blood vessel in the full-view setting (FIGS. 8A and 8D, respectively). For the limited-view settings, the US are weakly focused on the off-center branches of the blood vessels as shown in FIGS. 8B, 8C, 8E and 8F. Focusing is particularly poor for the structures oriented perpendicular to the transducer surface for the limited-view cases.

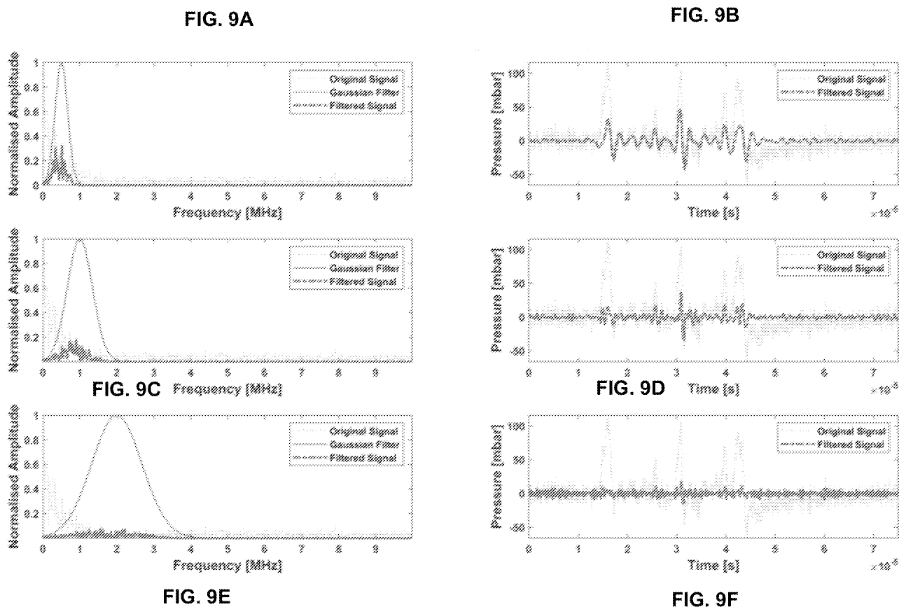

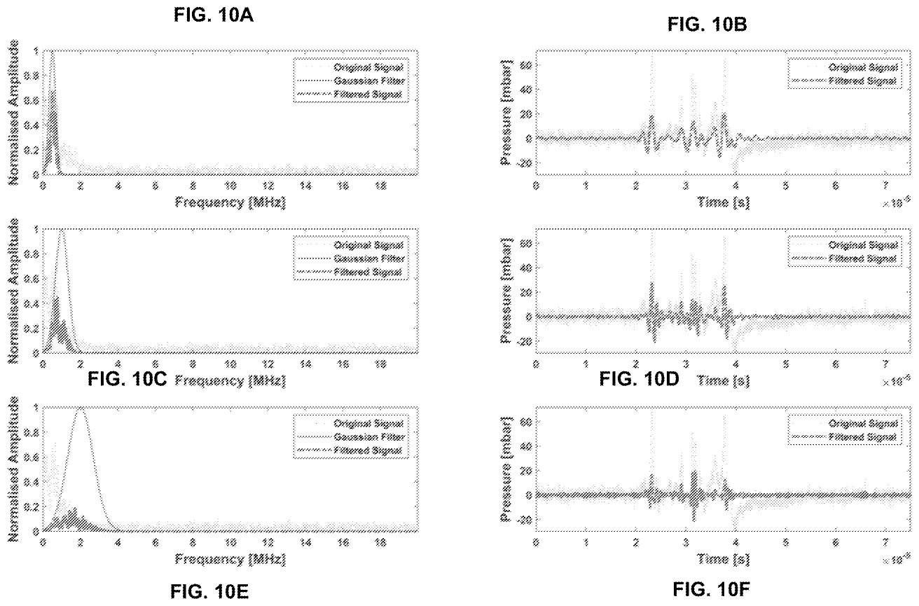

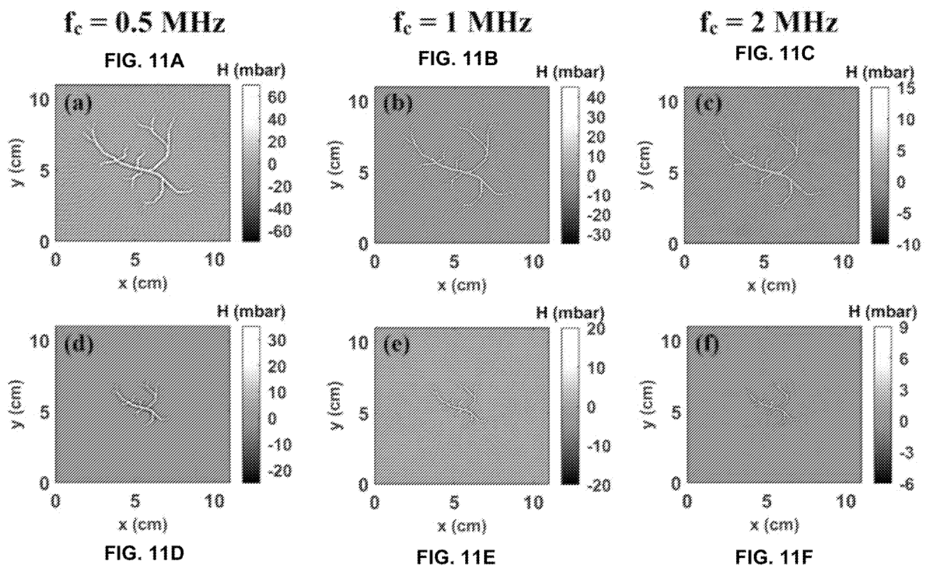

Another important factor to govern the efficacy of an example methodology according to embodiments is the bandwidth or the frequency response of the transducers. While the PA waves generated due to absorption of a short laser pulse have broadband frequency spectra (hundred kHz to tens of MHz), the frequency responses of the transducers are bandlimited, resulting in loss of the acoustic energy collected. Consequently, the magnitude of the focused US on the targeted blood vessel would also be smaller. In some examples simulated were the frequency response as Gaussian filters with central frequencies (fc) of 0.5 MHz, 1 MHz, and 2 MHz, with 80% bandwidth. The effect of detection response on the PA signals is demonstrated in FIGS. 9 and 10 for phantom 1 and 2 respectively and the converged US fields at 75 μs are displayed in FIG. 11.

For example, the converged US energy is more diffused for the transducers with low central frequency (FIGS. 11A and 11D) as compared to the ones with higher central frequencies (FIGS. 11B, 11C, 11E and 11F). As indicated in FIGS. 9A to 9F and FIGS. 10A to 10F, the US energy of the true PA signals from both the phantoms is primarily concentrated in the lower frequencies. Therefore, the magnitude of the focused ultrasound decreases with increasing transducer central frequency for both the phantoms as shown in FIGS. 11A to 11F. Another factor contributing to this is the acoustic attenuation in the medium, which as per Eq. 2, is stronger for the higher frequencies, thus further reducing the US energy contained in the higher frequency components. Also, the more the US travels in an acoustically attenuating media, the more it will attenuate. Therefore, as compared to phantom 1, the magnitude of the focused US irrespective of the transducer bandwidth, is weaker for phantom 2, which is located farther from the transducer grid. Choice of the transducer with consideration for an appropriate detection bandwidth is thus critical for the success of PWS treatment with the present embodiments. The blood vessels are typically thin (˜100 μm-1 mm) yielding high frequency PA signals. However, due to higher acoustic attenuation of the high frequency components, transducers with relatively lower central frequency should be preferred.

The computational studies performed by the present Applicant demonstrate the methodology and the efficacy of time-reversal guided US focusing for non-invasive PWS birthmarks treatment with minimal effect on neighboring tissue. The present embodiments could also benefit existing methodologies for ultrasound sonication which use microbubbles for rupturing blood vessels and for improved permeability for intravascular delivery of gene therapy and medications. The methodology can also assist other targeted ultrasound therapeutic techniques aiming wound healing and cancer therapy (Id.).

### Conclusion

Current standard of care pulsed dye laser-based treatment for port-wine stains is achieved by high-intensity laser excitation and subsequent thrombosis and destruction of blood vessels. However, in the process, a significant amount of optical energy is absorbed in the skin and the neighboring tissues which may be painful and cause thermal damage. Epidermal cooling methods and anesthesia are required to address these issues. The present embodiments relate to a photoacoustic guided US focusing methodology for PWS treatment. The PA signals collected by the transducers, when time-reversed and transmitted, converge onto the PWS, thus, minimally affecting neighboring tissue. The present Applicant performed simulations that mimic realistic transducers and medium properties for this proof of concept study demonstrating the feasibility of a methodology according to embodiments. The studies indicate that high coverage angle and element density of the transducer grid are crucial for achieving better convergence of the US field onto the targeted vessels. High elemental spacing in the detection grid leads to poorly focused US energy deposition on off-center targets and relatively higher energy deposition in neighboring tissue. Moreover, a transducer grid with low coverage angle causes poor US focusing on the blood vessels oriented perpendicular to the grid. Another governing factor for the efficacy of this methodology is choosing the transducer grid with an appropriate detection bandwidth. The sub-millimeter thick blood vessels produce high-frequency PA signals. However, due to higher acoustic attenuation of the high frequency components, transducers with relatively lower central frequency should be preferred. The present embodiments can also benefit other US therapeutic modalities such as sonication and increasing permeability for drug and gene delivery to blood vessels.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably coupleable,” to each other to achieve the desired functionality. Specific examples of operably coupleable include but are not limited to physically mateable and/or physically interacting components and/or wirelessly interactable and/or wirelessly interacting components and/or logically interacting and/or logically interactable components.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

Although the present embodiments have been particularly described with reference to preferred examples thereof, it should be readily apparent to those of ordinary skill in the art that changes and modifications in the form and details may be made without departing from the spirit and scope of the present disclosure. It is intended that the appended claims encompass such changes and modifications.

1. A method for photoacoustic (PA) guided ultrasound (US) focusing for port wine stain (PWS) treatment, comprising:
combining optical contrast-based selectivity with US penetration to focus the US energy onto PWS affected vasculature.

2. The method of claim 1, wherein the PA signals are collected by US transducers, time-reversed and US signals are transmitted onto the PWS affected vasculature so as to minimally affect neighboring tissue.

3. The method of claim 1, wherein the combining includes:
exciting tissue with laser signals to produce a PA excitation;
collecting a sinogram corresponding to the PA excitation;
performing a time-reversal on the collected sinogram to generate focusing information; and
focusing US signals onto the PWS affected vasculature using the focusing information.

4. The method of claim 3, wherein collecting the sinogram is performed using US transducers.

5. The method of claim 3, wherein collecting the sinogram includes determining one or both of a location and shape of the PWS affected vasculature.

6. The method of claim 2, further comprising adjusting a number and spacing of the US transducers to achieve a desired focusing.

7. The method of claim 2, further comprising adjusting a field of view of the US transducers to achieve a desired focusing.

8. The method of claim 2, further comprising adjusting a frequency bandwidth of the US transducers to achieve a desired focusing.

9. A device for photoacoustic (PA) guided ultrasound (US) focusing for port wine stain (PWS) treatment, comprising:
an optical component having contrast-based selectivity; and
a US component to direct US energy onto PWS affected vasculature using information generated by the optical component.

10. The device of claim 9, further comprising US transducers for collecting PA signals generated by the optical component.

11. The device of claim 9, further comprising a processor to time-reverse the collected PA signals.

12. The device of claim 9, wherein the optical component is a laser.

13. The device of claim 12, wherein the laser is a pulsed laser.

14. The device of claim 10, wherein a number and spacing of the US transducers is adjusted to achieve a desired focusing.

15. The device of claim 10, wherein a field of view of the US transducers is adjusted to achieve a desired focusing.

16. The device of claim 10, wherein a frequency bandwidth of the US transducers is adjusted to achieve a desired focusing.

17. The method of claim 4, further comprising adjusting a number and spacing of the US transducers to achieve a desired focusing.

18. The method of claim 4, further comprising adjusting a field of view of the US transducers to achieve a desired focusing.

19. The method of claim 4, further comprising adjusting a frequency bandwidth of the US transducers to achieve a desired focusing.

20. The device of claim 11, wherein the optical component is a laser.
